(19) United States
(12) Patent Application Publication
(10) Pub. No.: US 2010/0172951 A1
(43) Pub. Date: Jul. 8, 2010

(54) Enhanced Medical Implant

(76) Inventor: Ray Wasielewski, New Albany, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 12/348,280

(22) Filed: Jan. 3, 2009

(51) Int. Cl. A61K 9/00; A61P 43/00

(52) U.S. Cl. 424/423

(57) ABSTRACT

A medical implant comprising in combination a synthetic, biological, or autologous matrix (or scaffold) and pluripotent stem cells derived from human teeth. Stem cells are harvested from the dental pulp of extracted wisdom teeth. After the stem cells are removed, the hard tooth is ground into a base material for the manufacture of a porous matrix bone into which the stem cells can be added. Additionally, soft tissue from the harvested tooth may be used as a carrier scaffold for soft tissue applications such as meniscal or cartilage repair.

### BACKGROUND OF THE INVENTION

Stem cells are cells found in most, if not all, multi-cellular organisms. They are characterized by the ability to renew themselves through mitotic cell division and differentiating into a diverse range of specialized cell types. The functional definition of a stem cell is of a mammalian cell that retains the ability to regenerate tissue over a lifetime. Properties of stem cells can be illustrated in vitro, using methods such as clonogenic assays, where single cells are characterized by their ability to differentiate and self-renew.

The two broad types of mammalian stem cells are: embryonic stem cells that are found in blastocysts, and adult stem cells that are found in adult tissues.

In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues.

As stem cells can be grown and transformed into specialized cells with characteristics consistent with cells of various tissues such as muscles or nerves through cell culture, scientists are excited about their use in medical therapies.

The classical definition of a stem cell requires that it possess two properties: (1) Self-renewal—the ability to go through numerous cycles of cell division while maintaining the undifferentiated state; and (2) Potency—the capacity to differentiate into specialized cell types. In the strictest sense, this requires stem cells to be either totipotent or pluripotent. Pluripotent, embryonic stem cells can become any tissue in the body, excluding a placenta.

Embryonic stem cell lines are cultures of cells derived from the epiblast tissue of the inner cell mass (ICM) of a blastocyst or earlier morula stage embryos. A blastocyst is an early stage embryo—approximately four to five days old in humans and consisting of 50-150 cells. Embryonic stem cells are pluripotent and give rise during development to all derivatives of the three primary germ layers: ectoderm, endoderm and mesoderm. In other words, they can develop into each of the more than 200 cell types of the adult body when given sufficient and necessary stimulation for a specific cell type. Because of their combined abilities of unlimited expansion and pluripotency, embryonic stem cells remain a theoretically potential source for regenerative medicine and tissue replacement after injury or disease.

The term adult stem cell refers to any cell which is found in a developed organism that has two properties: the ability to divide and create another cell like itself and also divide and create a cell more differentiated than itself.

Pluripotent adult stem cells are rare and generally small in number but can be found in a number of tissues including umbilical cord blood. A great deal of adult stem cell research has focused on clarifying their capacity to divide or self-renew indefinitely and their differentiation potential.

Most adult stem cells are lineage-restricted (multipotent) and are generally referred to by their tissue origin (mesenchymal stem cell, adipose-derived stem cell, endothelial stem cell, etc).

Adult stem cell treatments have been successfully used for many years to treat leukemia and related bone/blood cancers through bone marrow transplants. Adult stem cells are also used in veterinary medicine to treat tendon and ligament injuries in horses. The use of adult stem cells in research and therapy is not as controversial as embryonic stem cells, because the production of adult stem cells does not require the destruction of an embryo. Additionally, because in some instances adult stem cells can be obtained from the intended recipient, (an autograft) the risk of rejection is essentially non-existent in these situations.

Ideally, one would use pluripotent adult stems cells from an autologous source, thereby removing the risk of rejection and obviating any political of moral qualms about the use of embryonic stem cells. Moreover these cells would demonstrate a level of pluripotency and genetic expression that equals that of embryonic stem cells.

It was not until recently that researchers have considered the possibility that stem cells in adult tissues could generate the specialized cell types of another type of tissue from that in which they normally reside. This facility of adult stem cells to generate specialized cell types of another type of tissue has been variously referred to as “plasticity,” “unorthodox differentiation,” or “transdifferentiation.” Presently, there is evidence that adult stem cells can generate mature, fully functional cells, or that the cells have restored lost function in vivo. Collectively, studies on plasticity suggest that stem cell populations in adult mammals are not fixed entities, and that after exposure to a new environment, they may be able to populate other tissues and possibly differentiate into other cell types.

Efforts are now underway to take advantage of the newly found capability of adult stem cells, with the goal of devising new treatments for disease and disability. Medical science is now providing voluminous evidence of many potential uses for stem cells, such as organogenesis, gene therapy, anti-aging therapies, angiogenesis, organ and tissue repair (particularly in cases of nerve damage), and the treatment of brain tumors, liver disease, and other diseases.

Adult stem cells from marrow may now be treated with certain chemicals such as dimethyl sulfoxide (DMSO) and hetastard with PBS, cryopreserved in liquid nitrogen, and later removed, thawed and used for transplantation and other therapies.

Today there is new evidence that adult stem cells may be found in more tissues and organs than previously thought, and that these cells are capable of developing into more kinds of cells than previously imagined. Efforts to devise new treatments for disease and disability utilizing adult stem cells hold great promise for the future if adult stem cells may be (i) secured from tissues of the body in a safe, painless, and convenient way, (ii) secured in acceptable quantity, (iii) isolated, (iv) propagated and aggregated (“expanded” via cellular division) to numbers useable for tissue regeneration, (v) and adapted to generate cell types of another type of tissue from that in which they normally reside.

Stem Cells: Scientific Progress and Future Research Directions (June 2001), indicate that sources of adult stem cells include bone marrow, peripheral blood, blood vessels, the cornea and the retina of the eye, brain, skeletal muscle, dental pulp, liver, skin, the lining of the gastrointestinal tract, and pancreas. Methods and apparatus have been developed to remove stem cells from some of these areas of the human body.

As to removal of stem cells from bone material specifically, such methods and apparatus include: U.S. Pat. Publication No. 20080176325 to Bowermaster, et al., the contents of which are incorporated here by reference.

Significantly, adult stem cells obtained from dental pulp may be a particularly good source of genetic material for gene therapy (in, e.g., the production of “genomic pharmaceuticals”), and in genotyping for donor registration and donor matching. Also, teeth are not affected by the soft tissue or hard tissue tumors of other parts of the body, including tumors of the jaw, even after metastasis of such tumors. Accordingly, it may be possible to take teeth from a patient prior to administering chemotherapy or radiotherapy in the treatment of cancer in other parts of the body.

It may then be possible to autograft the expanded adult stem cells and other cells harvested from those teeth back into the patient. By such a procedure, it may be possible to thereby reintroduce into the patient his or her own “fresh,” non-cancerous regenerative cells. Such cells, as with bone marrow “rescue” therapy, have not have been exposed to the damaging effects of chemotherapy and radiotherapy, and so are unaffected by such therapies. However, such cells are less likely to have tumorous or metastasized tissues which could then repopulate the patient.

Harvesting the dental pulp from a tooth requires dissecting or opening the tooth to reveal the pulp, or otherwise manipulating the pulp to remove it from the pulp chamber and root. Thereafter, the method of the present invention requires combining said pulp with an autologous biologic bone matrix or a synthetic biocompatible scaffold. In this respect, the method of the present invention substantially departs from pre-existing methods of the prior art, and all apparatus associated with methods of the prior art, and in so doing provides the user with a means for harvesting stem cells (and other cells which may be found in teeth and applied for medical purposes) from the pulp of teeth and combining the same with useful scaffold.

The gold standard of bone grafting is taking the patient's own bone. This is called autogenous bone graft. This generally means that at the time of surgery, the surgeon makes a separate incision and takes a small piece of bone from an area of the body where it is not needed. Typically, autogenous bone grafts are taken from the pelvis or iliac crest. Autogenous bone grafting has excellent fusion rates and has become the standard by which all other biologics are measured. Many surgeons prefer autogenous bone grafts because there is no risk of the body rejecting the graft since it came from the patient's own body.

There are disadvantages of autogenous bone grafting including the need for an additional incision, pain and soreness which often last well after the surgery is healed, as well as possible complications such as increased blood loss and prolonged time in the operating room. Complications such as these occur in about 10%-35% of patients and vary in their severity. Even when using a patient's own bone, 100% fusion rates are not always achieved, which is why other fusion techniques have been developed.

In an effort to minimize the problems associated with taking the patient's own bone, a number of other fusion techniques have been developed that use biological products as bone graft extenders or as bone graft replacements. One common source of bone graft replacement or extender is the use of allograft bone. An allograft bone graft is bone harvested from cadavers or deceased individuals who have donated their bone for use in the treatment of living patients. This is commonly used in many forms for spinal fusions ranging from cervical interbody fusions to lumbar interbody fusions and can provide excellent structural support.

The disadvantage of allograft structural bone is that it does not promote bone growth very well and therefore is very weak at stimulating a spinal fusion. Although it is used successfully for short-level fusions in the cervical spine, it is not a powerful enough biological stimulant to allow us to successfully use this to achieve a spinal fusion in the thoracic or lumbar spine. Studies have shown that when using allograft bone as the only graft material, the fusion rates in the thoracic and lumbar spine are extremely poor and the failure rate is very high.

In addition to hard implants that facilitate bone fusion, the implants of the present invention are usable combination with collagen as well.

Collagen has been utilized for a variety of clinical purposes including wound treatment, hemostasis, and soft tissue augmentation. Soluble collagen has been used as a subcutaneous implant for repairing dermatological defects such as acne scars, glabellar furrows, excision scars and other soft tissue defects.

The formulations of the invention provide tissue ingrowth through cell migration into the interstices of the collagen matrix. The very porous collagen matrix forms a skeleton providing sufficient volume for cells to attach and grow into the matrix, and to synthesize their own macromolecules. The cells thereby produce a new matrix which allows for the growth of new tissue.

Moreover, additives such as hyaluronic acid and fibronectin can be added to implants of the present invention. Hyaluronic acid in the collagen matrix encourages cellular infiltration into the pores and channels of the matrix. Fibronectin induces cell attachment to the collagen fibers of the matrix.

### SUMMARY OF THE INVENTION

In a first embodiment, the present invention relates generally to a medical implant composition comprising in combination a collection of pluripotent stem cells collected from the dental pulp of a harvested human tooth; and synthetic matrix formed from a material selected from the group consisting of a biocompatible metal, a biocompatible thermoplastic, a biocompatible thermoset, and a biocompatible composite.

In a further embodiment, the present invention relates generally to a medical implant composition comprising in combination, a collection of pluripotent stem cells collected from the dental pulp of a harvested human tooth; and an autologous biological matrix formed from a source selected from the group consisting of the harvested tooth, bone from an autologous donor, and soft tissue from the donor.

In another embodiment, the present invention relates to an allograft medical implant and a method of making the same comprising: removing a human tooth from a donor; harvesting pluripotent cells from the pulp of the tooth; cryogenically preserving the cells and the tooth; pulverizing the tooth to form a bone powder therefrom; forming a bone matrix from the tooth powder; and combining the cells and the matrix to form the implant.

The present invention further relates to a method of fabricating a medical implant comprising: removing a human tooth from a donor; harvesting pluripotent cells from the pulp of the tooth; cryogenically preserving the cells and the tooth; pulverizing the tooth to form a bone powder therefrom; forming an autologous bone matrix from the tooth powder; and combining the cells and the matrix to form the implant.

### BRIEF DESCRIPTION OF THE DRAWINGS

FIG. 1 is a drawing of a human tooth.

### DETAILED DESCRIPTION OF THE INVENTION

Several exemplary embodiments of the present invention shall be described below with reference to the drawings herein. However, those of ordinary skill in the art will understand that other embodiments not disclosed herein are still within the scope of this invention and its claims.

For example the devices and methods disclosed and claimed in the present invention are applicable to all manner of medical implants anatomies, including spine, knee, hip, shoulder, elbow, and the like.

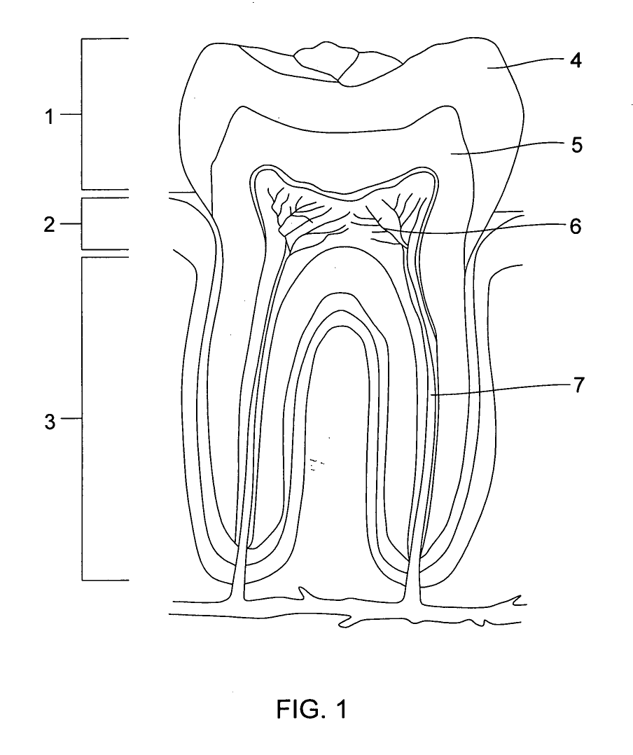

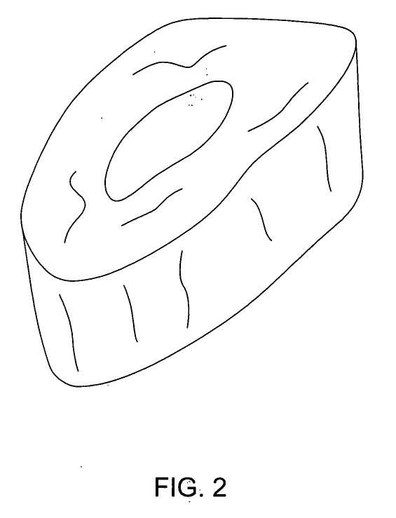

Referring first to FIG. 1, there is shown a drawing of the anatomy of a human tooth, which anatomy generally includes: the crown (1); the neck (2); the root (3); the enamel (4); the dentin (5); pulp cavity (6); and root canal (7). It has been shown that pluripotent stem cells can be found in the pulp area of human teeth, specifically the pulp area of unerupted wisdom teeth, and harvested therefrom. One such method for accomplishing such harvesting is disclosed in U.S. Patent Publication 20080176325 to Bowermaster, et al., the disclosure of which is incorporated herein.

The primary difficulties encountered with stem cells from sources other than dental pulp has been a limited number of cells and difficulty culturing and growing the isolated cells. Two to five milliliters of amniotic fluid, for example, have been reported to contain approximately 1-2×104 live cells per milliliter. However, dental pulp may contain millions of stem cells. These cells are relatively homogeneous and exhibit markers associated with embryonic stem cells. The isolated tissue also comprises cells that are relatively easy to culture and reproduce.

Such cells can be readily obtained by isolating a mesenchymal dental papilla from an un-erupted tooth such as, for example, an un-erupted third molar. As used herein, “isolated” or “isolating” refers to removal of the tissue mass containing stem cells from the oral cavity of a mammal, especially a human. For each such un-erupted tooth, a tissue mass can be isolated to provide up to about 8 to 12 million cells per tooth.

These cells can be obtained by isolating the developing dental pulp, or dental papilla or any preerupted permanent tooth in a mammal. A tooth bud is a knoblike primordium that develops into an enamel organ surrounded by a dental sac, encasing the dental papilla. Dental papilla is a mass of mesenchymal tissue that ultimately differentiates to form dentin and dental pulp. The dental sac ultimately differentiates to form the periodontal ligament. Tooth buds appear in early childhood, with the last, the third molar, beginning to form at approximately four years of age in a human child. By the time the twenty deciduous teeth have erupted, the first permanent molars are also erupted or erupting, and there are approximately 28 tooth buds for permanent teeth in various states of development in the tissue beneath the deciduous teeth. By the time the teeth erupt, the enamel organ has generally encased the dental pulp. Prior to eruption, however, the mesenchymal tissue may be surgically removed to provide an isolated tissue comprising millions of stem cells, and any tooth bud or un-erupted tooth may provide an isolated tissue according to the present invention.

A particularly attractive source of isolated tissue is the un-erupted third molar, since these developing teeth are often surgically removed because there is insufficient room in the oral cavity for them to erupt or they are not developing normally and may force other teeth out of alignment if they are not removed. Third molars, often called “wisdom teeth”, generally erupt between the ages of 17 and 21. Second molars usually erupt between the ages of 11 and 13, and third molars may be detected by x-ray at about this time. If there is not sufficient room for the third molar or it is not developing normally (e.g., some third molars appear to be growing “sideways” in maxilla or mandible), the molar may be surgically removed at this point so that it cannot become impacted (which may occur if the developed tooth has not reached its appropriate final position by adulthood) or produce misalignment of the other teeth as it develops.

Third molars are customarily removed from pre-teen and teenage patients while the teeth are still developing, and while the primordial bulb still contains millions of stem cells. Since approximately 800,000 third molars (generally the last set of teeth to erupt) are removed each year in the United States alone, and the inventors have demonstrated that each of these teeth comprises an associated tissue mass that contains approximately 8 to 12 million cells per tooth, removal of four third molars from one individual may provide a minimum of approximately 20 million multipotent stem cells. A majority of these cells have been shown to be Oct4 positive, SSEA1 positive, SCAI negative, MART-I negative, TRA80-1 positive, SS EA-4 negative, CD 117 negative and TRA60-1 negative, indicating that the cells are primitive, multipotent stem cells that may be induced to differentiate into a variety of cell and tissue types.

One method of the present invention comprised forming a bone graft using a human tooth or human teeth as the primary component of the graft matrix. Persons of skill in the art will understand that the same method can be applied to non-human mammals and reptiles, allograft bone sources, or even synthetic sources of scaffold. Moreover, the present invention may be used to replace soft tissue, such as collagen, in addition to or instead of bone.

The steps of the forming a bone graft or other medical implant comprise: obtaining source bone material from a donor; forming the source bone into a desirable shape; and sterilizing the graft. Moreover, the method of the present invention includes the additional step of combining stem cells collected from an unerupted tooth or teeth from a living or deceased human donor, preferably in the age range of from about 13, to 22.

While one may use any human bone to make the structural portion of the of the medical implant of the present invention, there are several potential advantages of using human teeth as the primary material. These advantages include lowering the risk of rejection, abundant supply of stem cells from unerupted human teeth, and low to no risk of cancer or other diseases in the stem cells or hard tooth.

The method of the present invention includes several alternative embodiments, including using a single autologous donor, multiple donors, or animal donors.

Those of skill in the art will recognize that use of a single autologous donor for the stem cells and the bone graft source has the advantage of eliminating the risk of rejecting from the medical implant.

Of course, multiple donors can also be used within the scope of the present invention. Where multiple donors are used as the source of either the stem cells or the bone graft, said donors and patients can be matched using standard known matching means, such as human leukocyte antigen (HLA) typing. HLA typing is used to match patients and donors for bone marrow or cord blood transplants (also called BMT). HLA are proteins—or markers found on most cells in your body. One's immune system uses these markers to recognize which cells belong in his/her body and which do not.

A close match between a patient's HLA markers and his/her donor's can reduce the risk that the patient's immune cells will attack the donor's cells or that the donor's immune cells will attack the patient's body after the transplant. If one needs an allogeneic transplant (which uses cells from a family member, unrelated donor or cord blood unit), a doctor will take a blood sample to test for the patient and donor for HLA type

A close HLA match improves the chances for a successful allograft implant. Close matching promotes engraftment. Engraftment is when the donated cells start to grow and make new blood cells, and reduces the risk of a post-transplant complication called graft-versus-host disease (GVHD). GVHD occurs when the immune cells from the donated marrow or cord blood (the graft) attack your body (the host).

Other embodiments of the present invention combine stem cells collected from dental pulp as described above in combination with a synthetic scaffold such as a porous biocompatible metal like porous tantalum, or porous titanium, both of which are presently commercially available.

In still further embodiments, the method of the present invention comprises adding certain known releasable antibacterial or antifungal compounds to the medical implant to discourage infection at the implant location. These additives include standard antibiotic medicines such as penicillin, and anti microbial metal alloys of copper or silver.

Possible antifungal medicines include: econazole, fenticonazole, miconazole, sulconazole, tioconazole, amphotericin, nystatin, terbinafine, itraconazole, fluconazole, ketoconazole, and griselfulvin, all of which come in various different brand names.

Sometimes antifungal medicines are combined with other medicines when two actions are required. For example, an antifungal is often combined with a mild steroid such as hydrocortisone to treat certain problems. The antifungal clears the infection, and the mild steroid reduces the inflammation caused by the infection.

Other embodiments or the present invention further include using bone growth promoting additives such as bisphosphonates releasably bonded to the implant. Exemplary coating methods are disclosed in U.S. Patent Publication No. 20060188542A and U.S. Pat. No. 7,163,690. Bisphosphonates are antiresorptive medicines. This means that they slow or stop the natural process that dissolves bone tissue, resulting in maintained bone density and strength. This may prevent the development of osteoporosis. If osteoporosis already has developed, slowing the rate of bone thinning reduces the risk of broken bones. Specific, bisphosphonates include alendronate, etidronate, ibandronate, risedronate, and zolendronic acid.

Still further embodiments of the present invention include using platelet gel or autologous platelet get to increase the volume of cells used in the medical implant. Platelets play an important role in wound healing. They provide initial control of bleeding (hemostasis) and they release mediators to help modulate the inflammatory response and many of the cellular functions involved in wound healing. Platelet Get is a substance that is created by harvesting platelet-rich plasma (PLZP) from whole blood and combining it with thrombin and calcium or other activators to form a coagulum. This coagulum or “platelet gel” has an extremely wide-range of clinical healing uses from dental surgery to orthopedics and plastic surgery. Over the past 10 years, scientists have discovered a lot of information about stem cells, growth factors, platelets and white cells, natural healing factors that circulate in your blood. These can be safely and efficiently harvested at a high enough concentration to be of therapeutic benefit when placed in a surgical wound.

Of course, any of a variety of known methods of making an allograft or autograft may be used with the present invention. These include, for example, the methods disclosed in U.S. Pat. Nos. 6,511,509 and 7,018,412 the disclosures of which are incorporated herein by reference.

It is to be understood that the scope of the present invention is not to be limited to the specific embodiments described above. The invention may be practiced other than as particularly described and still be within the scope of the accompanying claims.

What is claimed is:

1. A medical implant for a human patient, comprising:
a. a collection of pluripotent stem cells harvested from the dental pulp of a human tooth, said cells embedded in
b. a synthetic matrix formed from a material selected from the group consisting of a biocompatible metal, a biocompatible thermoplastic, a biocompatible thermoset, and a biocompatible composite.

2. A medical implant for a human patient, comprising:
a. a collection of pluripotent stem cells harvested from the dental pulp of a human tooth, said cells embedded in
b. an autologous biological matrix formed from a source selected from the group consisting of the harvested tooth, bone from an autologous donor, and soft tissue from the donor.

3. A medical implant for a human patient, comprising:
a. a collection of pluripotent stem cells harvested from the dental pulp of a plurality of human teeth, said cells embedded in
b. a bone graft formed from the plurality a human teeth.

4-18. (canceled)

19. A medical implant according to claim 1, wherein the implant is shaped for implantation into a human tibia.

20. A medical implant according to claim 1, wherein the implant is shaped for implantation into a human distal femur.

21. A medical implant according to claim 1, wherein the implant is shaped for implantation into a human vertebra.

22. A medical implant according to claim 1, wherein the implant is shaped for implantation into a human proximal femur.

23. A medical implant according to claim 2, wherein the implant is shaped for implantation into a human tibia.

24. A medical implant according to claim 2, wherein the into a human distal femur.

25. A medical implant according to claim 2, wherein the implant is shaped for implantation into a human vertebra.

26. A medical implant according to claim 2, wherein the implant into a human proximal femur.

27. A medical implant according to claim 3, wherein the implant into a human tibia.

28. A medical implant according to claim 3, wherein the implant into a human distal femur.

29. A medical implant according to claim 3, wherein the implant is shaped for implantation into a human vertebra.

30. A medical implant according to claim 3, wherein the implant is shaped into a human proximal femur.

31-46. (canceled)

47. A medical implant according to claim 1, wherein the stem cells show a higher level of genetic expression than reproduced stem cells.

48. A medical implant according to claim 1, wherein the stem cells have a level of pluripotency greater than stem cells from umbilical cords.
